(19) United States
(12) Patent Application Publication
(10) Pub. No.: US 2009/0069873 A1
(43) Pub. Date: Mar. 12, 2009

(54) Transparent film for a phototherapy device

(76) Inventor: Hendrik J. Vreman, Los Altos, CA (US)

Correspondence Address: LUMEN PATENT FIRM, 2345 YALE STREET, SECOND FLOOR, PALO ALTO, CA, 94306, US

(21) Appl. No.: 12/231,068

(22) Filed: Aug. 27, 2008

Related U.S. Application Data
(60) Provisional application No. 60/966,580, filed on Aug. 28, 2007.

(51) Int. Cl. A61N 5/06

(52) U.S. Cl. 607/90

(57) ABSTRACT

Transparent films for efficient phototherapy treatment of a patient and reducing phototherapy light exposure to non-patients are provided. Patients undergoing hyperbilirubinemia generally require phototherapy treatment, which can cause undesirable symptoms to non-patients, including caregivers, in the vicinity of the treatment area. Flexible transparent films are provided to at least partially reflect and/or absorb the phototherapy light, thereby providing comfort to the non-patients. Due to the at least partial transparency of the films, a non-patient can visually observe the patient to provide care to the patient. In addition, the transparent films can reflect the light down to the patient for increasing the phototherapy treatment efficacy.

### CROSS-REFERENCE TO RELATED APPLICATIONS

This application claims priority from U.S. Provisional Patent Application 60/966,580 filed Aug. 28, 2007 which is incorporated herein by reference.

### FIELD OF THE INVENTION

The present invention relates generally to phototherapy devices. More particularly, the present invention relates to transparent films to improve the comfort of non-patients while being in the treatment room by reducing or eliminating the light exposure caused by the phototherapy device and/or to improve the effectiveness of phototherapy.

### BACKGROUND

Phototherapy with intense blue light for the treatment of patients with hyperbilirubinemia (jaundice) results in the emission of (intense) blue light into the nursing environment. Up to 50% of nursing staff and/or family members exposed to this blue light report various unpleasant symptoms such as seeing yellow spots, headache, nausea, dizziness, and mild vertigo. These symptoms cause distress in persons needing to work in close proximity to these treatment devices. Methods traditionally used to counteract these symptoms have a number of drawbacks, such as reduced airflow to the patient, and reduced visibility of the patient.

Another problem with traditional phototherapy devices is that much of the therapeutic light is not directed to the patient and therefore wasted. For example, stray light is lost into the environment.

Accordingly, there is a need in the art to develop new methods for increasing the effectiveness of phototherapy and the comfort of non-patients in the treatment room.

### SUMMARY OF THE INVENTION

The present invention is directed to transparent films for efficient phototherapy treatment for patients and comfort for non-patients. In particular, the present invention is directed to a method of reducing or eliminating the exposure of light generated by a phototherapy device to one or more non-patients present in a treatment room when a patient is treated with the phototherapy device in the treatment room. The method includes having a phototherapy device in the treatment room and providing one or more flexible transparent films attached, affixed, or placed in proximity to the phototherapy device, wherein the flexible transparent films reflect and/or absorb at least some of the light generated by the phototherapy device for reducing or eliminating the light exposure to one or more non-patients, and wherein the flexible transparent films are sufficiently transparent to allow one or more of the non-patients in the treatment room to visually observe the patient through the flexible transparent films.

In a preferred embodiment, the patient has hyperbilirubinemia, the phototherapy device emits light with a wavelength in the range of about 400 nm to about 520 nm, the flexible transparent films absorb or reflect light with wavelengths in the range of about 400 nm to about 520 nm, and the flexible transparent films are optically transparent to visible light with wavelengths above about 520 nm. In another embodiment, the flexible transparent film absorbs greater than about 90% of the light generated by the phototherapy device. In yet another embodiment, the light is reflected by the flexible transparent films for increasing the light exposure to the patient, whereby increasing the efficacy of the phototherapy on the patient.

As mentioned above, the flexible transparent film reduces the light exposure to one or more of the non-patients in the treatment room. In particular, the reduction of the light exposure is associated with the prevention of symptoms, such as seeing yellow spots, headache, nausea dizziness, and mild vertigo, to the non-patients.

In another embodiment, one or more of the flexible transparent films can be detached, folded, hinged or rolled to provide access to the patient. The flexible transparent films can also allow airflow in and out of the area between the flexible transparent films and the phototherapy device.

### BRIEF DESCRIPTION OF THE FIGURES

The present invention together with its objectives and advantages will be understood by reading the following description in conjunction with the drawings, in which:

FIG. 1 shows an example of transparent films for a phototherapy device according the present invention.

FIG. 2 shows an example of a transparent film for a suitcase phototherapy device according to the present invention.

### DETAILED DESCRIPTION OF THE INVENTION

The objectives of the invention are: (i) improving the comfort of a non-patient present in a treatment room caused by light generated by a phototherapy device when a patient is treated with the phototherapy device, and/or (ii) increasing the effectiveness (light delivery or exposure) of a phototherapy when a patient is treated with the phototherapy device. In the case where the invention is used to improve the comfort of the non-patient, the present invention may reduce symptoms in the non-patient including but not limited to seeing yellow spots, headache, nausea, dizziness, and mild vertigo. In the case where the invention is used to increase efficacy of phototherapy, advantage is taken of light that would not normally directly impact the patient. This would include any light that is not directed at the patient.

In an embodiment of the present invention, the patient is a person undergoing phototherapy treatment for hyperbilirubinemia and the non-patient is any of the other people in the treatment room where the phototherapy device is located and is not being treated with the phototherapy device. It is noted that any patient requiring phototherapy and/or any non-patient may benefit from this invention.

To achieve the objectives of this invention, one or more transparent films are included with the phototherapy device that either reflect and/or absorb the light at the same wavelength as generated by the phototherapy device. The transparent film could also be transparent to other wavelengths of light.

In one embodiment, the phototherapy device emits light with a wavelength in the range of about 400 nm to about 520 nm. Accordingly, the transparent film absorbs or reflects light with wavelengths in the range of about 400 nm to about 520 nm. Preferably, the transparent film could be optically transparent to visible light with wavelengths above about 520 nm.

The transparent film could be made of any transparent material, including but not limited to plastics such as polycarbonate or mylar. Importantly, the film should be of sufficient transparency to allow uninterrupted observation of the patient by the non-patient. Preferably, the film is greater than 70% optically transparent, more preferably 80% transparent, even more preferably 90% transparent, and most preferably 100% transparent to wavelengths outside the range of wavelengths used by the phototherapy device for treatment of the patient. The film is preferably non-rigid, i.e. flexible.

In an embodiment in which the transparent film has absorbent features, it could absorb at least 70% (more preferably, 80%, even more preferably 90%, and most preferably 100%) of the light generated by the phototherapy device. In an embodiment in which the transparent film is reflective, it preferably reflects at least 75% (more preferably 85%, and most preferably 100%) of the light generated by the phototherapy device. Preferably, the transparent film is greater than 60% (more preferably 80%, and most preferably 100%) optically transparent to wavelengths outside the range of wavelengths used by the phototherapy device for treatment of the patient or used by the room light.

The transparent film could be attached or affixed to the phototherapy device or positioned near (or in proximity with) the phototherapy device. Examples for affixing the transparent film to the phototherapy device include, but are not limited to, a hook and loop system, one or more magnets, adhesive tape, or other types of fasteners. In addition, any number, combination, and type of film (i.e. absorbing and/or reflecting) may be used. For example, for a phototherapy device with four sides, any combination of one through four absorbent and/or reflective transparent films may be used. In an exemplary embodiment, a transparent film could be fixed along at least one side of the phototherapy device regardless of the specific device used. The transparent film could also be modified to function with a specific phototherapy device. Furthermore, the transparent film could be fixed in such way as to allow air to flow into and out of the phototherapy device.

Preferably, the transparent film may be removed to provide access to the patient. This can be accomplished in various ways. For example, the transparent film may be detached from the phototherapy device. Alternatively, the film may be rolled, folded, or hinged along one end.

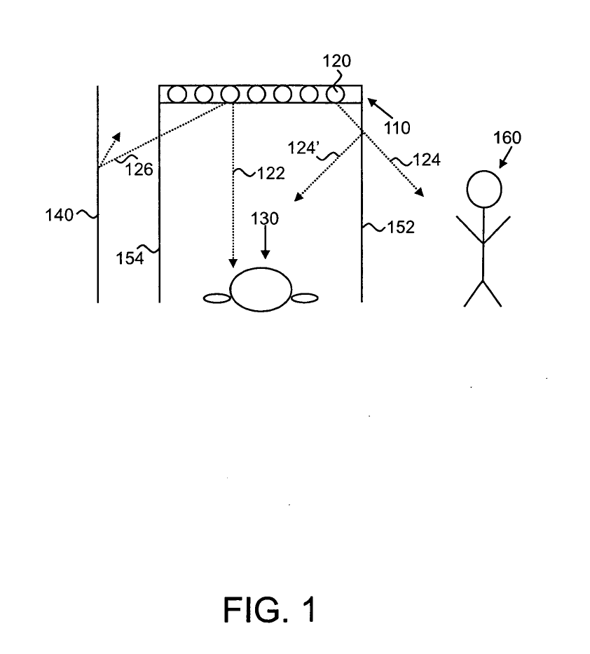

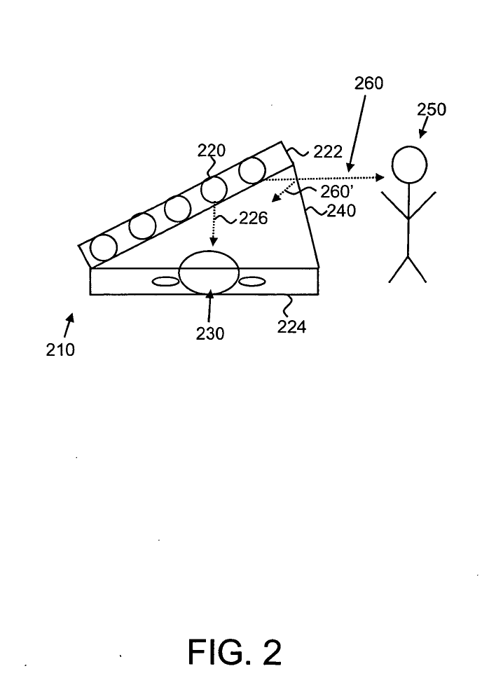

The following sections describe some examples of embodiments. FIGS. 1-2 show examples that illustrate implementations of the present invention. These are exemplary only and should not be taken as limiting.

FIG. 1 shows a phototherapy device 110 with a plurality of light sources 120. Phototherapy device 110 is suspended over patient 130 (possibly) near a wall 140 to treat patient 130 with therapeutic light 122. Transparent films 152 and 154 are fixed to opposing sides of phototherapy device 110. Transparent film 152 serves to protect non-patient 160 from receiving stray light 124 from light sources 120. Transparent film 154 protects non-patient 160 from receiving stray light 126 that could reflect off of wall 140. Alternatively or additionally, transparent films 152 and 154 may reflect light back onto patient 130, thereby increasing the efficacy of phototherapy device 110. An example of such a reflection of stray light back to the patient is shown by reflection 124′.

FIG. 2 shows a suitcase-type phototherapy device 210 with a plurality of light sources 220 in a housing 222. Housing 222 is hinged to bed 224, in which patient 230 lies. Phototherapy device 210 treats patient 230 by emitting therapeutic light 226 from light sources 220. Transparent film 240 is attached, fixed, or positioned proximate to housing 222 and/or bed 224. Transparent film 240 serves to protect non-patient 250 from receiving stray light 260 from light sources 220. Alternatively or additionally, transparent film 240 may reflect light back onto patient 230, thereby increasing the efficacy of phototherapy device 210. An example of such a reflection of stray light back to the patient is shown by reflection 260′.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. A method of reducing or eliminating the exposure of light generated by a phototherapy device to one or more non-patients present in a treatment room when a patient is treated with said phototherapy device in said treatment room, said method comprising:
a) having a phototherapy device in said treatment room; and
b) providing one or more flexible transparent films attached, affixed, or placed in proximity to said phototherapy device, wherein said flexible transparent films at least partially enclose said patient from said non-patient, wherein said one or more flexible transparent films reflect, absorb, or reflect and absorb at least some of said light generated by said phototherapy device, and wherein said one or more flexible transparent films are sufficiently transparent to allow one or more of said non-patients present in said treatment room to visually observe said patient through said one or more flexible transparent films,
wherein the reflected or absorbed light by said one or more flexible transparent films results in the reduction or elimination of said light exposure to said one or more non-patients in said treatment room.

2. The method as set forth in claim 1, wherein said patient has hyperbilirubinemia, wherein said phototherapy device emits light with a wavelength in the range of about 400 nm to about 520 nm, wherein said one or more flexible transparent films absorb or reflect light with wavelengths in the range of about 400 nm to about 520 nm, and wherein said one or more flexible transparent films are optically transparent to visible light with wavelengths above about 520 nm.

3. The method as set forth in claim 1, wherein said one or more flexible transparent films absorb greater than 90% of said light generated by said phototherapy device.

4. The method as set forth in claim 1, wherein the reflected light by said one or more flexible transparent films results in increasing the light exposure on said patient, whereby increasing the efficacy of said phototherapy on said patient.

5. The method as set forth in claim 1, further comprising detaching, folding, hinging, or rolling one or more of said one or more flexible transparent films to provide access to said patient.

6. The method as set forth in claim 1, wherein said reduction of said light exposure to said one or more non-patients in said treatment room is associated with the prevention of symptoms to said one or more non-patients, wherein said symptoms include at least one of seeing yellow spots, headache, nausea, dizziness, and mild vertigo.

7. The method as set forth in claim 1, wherein said one or more flexible transparent films allow airflow in and out of the area between said one or more flexible transparent films and said phototherapy device.

8. A method of increasing the effectiveness of a phototherapy on a patient, comprising:
a) having a phototherapy device in said treatment room; and
b) providing one or more flexible transparent films attached, affixed or placed in proximity to said phototherapy device in such a way to at least partially enclose said patient, wherein said one or more flexible transparent films reflect said light generated by said phototherapy device and wherein said one or more flexible transparent films are sufficiently transparent to allow one or more non-patients present in a treatment room to visually observe said patient through said one or more flexible transparent films,
wherein the reflected light by said one or more flexible transparent films results in increasing the effectiveness of said phototherapy on said patient or results in increasing the light exposure on said patient.

9. The method as set forth in claim 8, wherein said patient has hyperbilirubinemia, wherein said phototherapy device emits light with a wavelength in the range of about 400 nm to about 520 nm, wherein said one or more flexible transparent films reflect light with wavelengths in the range of about 400 nm to about 520 nm, and wherein said one or more flexible transparent films are optically transparent to visible light with wavelengths above about 520 nm.

11. The method as set forth in claim 8, further comprising detaching, folding, hinging, or rolling one or more of said one or more flexible transparent films to provide access to said patient.

12. The method as set forth in claim 8, wherein said one or more flexible transparent films result in reducing or eliminating the light exposure to said one or more non-patients in said treatment room.

13. The method as set forth in claim 12, wherein said reducing or eliminating the light exposure to said one or more non-patients in said treatment room is associated with the prevention of symptoms to said one or more non-patients, wherein said symptoms include at least one of seeing yellow spots, headache, nausea, dizziness, and mild vertigo.

14. The method as set forth in claim 8, wherein said one or more flexible transparent films allow airflow in and out of the area between said one or more flexible transparent films and said phototherapy device.
